(19) United States
(12) Patent Application Publication
(10) Pub. No.: US 2006/0030940 A1
(43) Pub. Date: Feb. 9, 2006

(54) Use of autogenous growth factors in bone tunnels during ligament reconstruction with mechanical containment implants

(76) Inventor: Reinhold Schmieding, Naples, FL (US)

Correspondence Address: DICKSTEIN SHAPIRO MORIN & OSHINSKY LLP, 2101 L Street, NW, Washington, DC, 20037, US

(21) Appl. No.: 11/183,851

(22) Filed: Jul. 19, 2005

Related U.S. Application Data
(60) Provisional application No. 60/589,159, filed on Jul. 20, 2004.

(51) Int. Cl. A61F 2/08

(52) U.S. Cl. 623/13.14

(57) ABSTRACT

A method for introducing autogenous growth factors produced from a patient's own blood into a target bone tunnel created by the surgeon during ligament reconstruction. The autogenous growth factors, preferably contained in platelet-rich plasma, are inserted into the target bone tunnel subsequent to the graft insertion to enhance the healing of the implant or graft and to promote bone growth.

This application claims the benefit of U.S. Provisional Application Ser. No. 60/589,159, filed on Jul. 20, 2004, the entire disclosure of which is incorporated by reference herein.

### FIELD OF THE INVENTION

The present invention relates to methods of reconstruction surgery and, in particular, to a method of ligament reconstruction using autogenous growth factors.

### BACKGROUND OF THE INVENTION

Methods of ligament reconstruction in the knee are known in the art. Reconstruction of anterior cruciate ligament, for example, is described in U.S. Pat. Nos. 5,211,647 and 5,320,626 and typically involves drilling a tunnel through the tibia, drilling a closed tunnel (socket) into the femur, inserting a substitute graft from one tunnel to the other, and securing the respective ends of the graft to the walls of the tibial and femoral tunnels using interference screws or transverse pins.

Although the existing method of ligament reconstruction described above is now widely practiced, it nevertheless has a number of attendant disadvantages. For example, extensive transosseous tunnels may be created to position replacement grafts in anatomical positions to reproduce the function of the damaged or absent ligament. Biologic incorporation of the replacement graft in the tunnel and the subsequent healing of the tunnel require an extensive period of immobilization, reduced patient activity levels, loss of motion and delayed return to normal daily functions during the lengthy healing process. Post-operatory loosening or lengthening of the graft may also occur.

### SUMMARY OF THE INVENTION

The present invention provides a method for introducing autogenous growth factors produced from a patient's own blood into a target bone tunnel created by the surgeon during ligament reconstruction. The autogenous growth factors, preferably contained in platelet-rich plasma (PRP), enhance the healing of the implant or graft provided within the target bone tunnel and promote bone growth.

The invention provides a method of conducting a ligament reconstruction employing autogenous growth factors, preferably contained in platelet-rich plasma (PRP), in a surgical procedure for which the growth of bone and/or tissue structure is promoted. The method comprises the steps of: (i) providing a target tunnel for ligament reconstruction; (ii) inserting a graft into the target tunnel; (iii) inserting autogenous growth factors, preferably contained in platelet-rich plasma (PRP), into the target tunnel; (iv) plugging the target tunnel with a synthetic plug, bone plug or screw to contain the PRP in the tunnel and accelerate healing, while also securing the graft in the tunnel.

In a preferred embodiment, the graft is secured into the target tunnel by employing two cannulated interference screws, each provided at one end of the target tunnel, plugging one of the two cannulated interference screws located at one end of the tunnel, inserting the growth factor enriched PRP into the tunnel through the cannulation of the unplugged screw, and then plugging the screw to contain the PRP in the tunnel during healing.

These and other features and advantages of the invention will be more apparent from the following detailed description that is provided in connection with the accompanying drawings and illustrated exemplary embodiments of the invention.

### BRIEF DESCRIPTION OF THE DRAWINGS

FIG. 1 illustrates a tibial tunnel provided with a graft and injected with autogenous growth factors in accordance with a method of the present invention.

FIG. 2 illustrates a cannulated interference screw which is plugged to prevent leakage of growth factors from the tibial tunnel.

### DETAILED DESCRIPTION OF THE PREFERRED EMBODIMENT

In the following detailed description, reference is made to various specific embodiments in which the invention may be practiced. These embodiments are described with sufficient detail to enable those skilled in the art to practice the invention, and it is to be understood that other embodiments may be employed, and that structural and logical changes may be made without departing from the spirit or scope of the present invention.

The term “endoscopy” encompasses arthroscopy, laparoscopy, hysteroscopy, among others, and endoscopic surgery involves the performance of surgical procedures within a patient's body through small openings as opposed to conventional open surgery through large incisions.

The term “growth factor” as used in the present application is intended to include all factors, such as proteinaceous factors, for example, which play a role in the induction or conduction of growth of bone, ligaments, cartilage or other tissues associated with bone or joints. In particular, these growth factors include bFGF, aFGF, EGF (epidermal growth factor), PDGF (platelet-derived growth factor), IGF (insulin-like growth factor), TGF-β I through III, including the TGF-β superfamily, VEGF, BMP 1 through 12, and GDF 1 through 12.

In a preferred embodiment, however, the term “growth factor” includes autogenous growth factors produced from a patient's own blood, preferably contained in platelet-rich plasma (PRP) obtained by a centrifugation process. In an exemplary embodiment, platelet-rich plasma (PRP) is prepared from a relatively small sample of patient's body by multiple-step centrifugation. In this manner, at the end of the centrifugation process, an autologous platelet concentrate is obtained which is rich in growth factors. In an exemplary embodiment, the autologous platelet concentrate is introduced into a target site, for example, into a bone tunnel provided with a graft during reconstructive knee surgery, and then sealed within the tunnel by fixation devices. The fixation devices prevent leaking of the autologous platelet concentrate from the target tunnel and promote the growth of bone and/or tissue structure and the overall healing process.

In an exemplary embodiment, PRP is prepared using a commercially available PRP concentration kits such as the SmartPReP® 2APC+ Platelet Concentration System sold by Harvest Technologies or the Symphony Platelet Concentration System sold by DePuy. Optionally, the resultant PRP, enriched with growth factors, may be mixed with a viscous substance, such as hyaluronic acid, to increase its viscosity and to ensure its adhesion to the target tunnel and graft.

The present invention provides a method for introducing autogenous growth factors produced from a patient's own blood into a target bone tunnel created by a surgeon during ligament reconstruction. Autogenous growth factors, preferably contained in platelet-rich plasma (PRP), are provided within the target bone tunnel subsequent to the insertion of the graft or ligament within the target bone tunnel and after securing both ends of the target tunnel with fixation devices, for example, cannulated interference screws. After one of the cannulated interference screws is plugged at one end of the target tunnel, autogenous growth factors are injected into the target tunnel through the unplugged cannulated interference screw located at the other end of the tunnel. The fixation devices prevent leaking of the autogenous growth factors from the target tunnel and promote the growth of bone and/or tissue structure and the overall healing process.

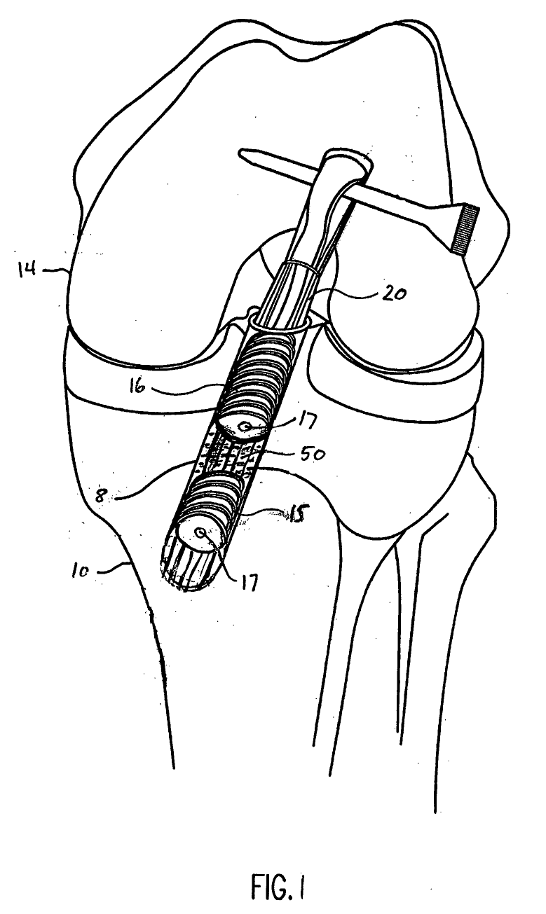

Referring now to the drawings, where like elements are designated by like reference numerals, FIG. 1 illustrates a graft 20 secured at one end within a tibial tunnel 8 of the tibia 10. FIG. 1 shows the other end of graft 20 secured in the femur 14 using a transverse pin, although it could also be secured in the femur with another interference screw (not shown), as is well known in the art. Referring back to the tibial side, interference screws 15 and 16 are secured between the graft 20 and the walls of the tibial tunnel 8. Interference screws 15 and 16 are cannulated, as is customary in the art, to allow them to be guided into the tunnel over a guide pin.

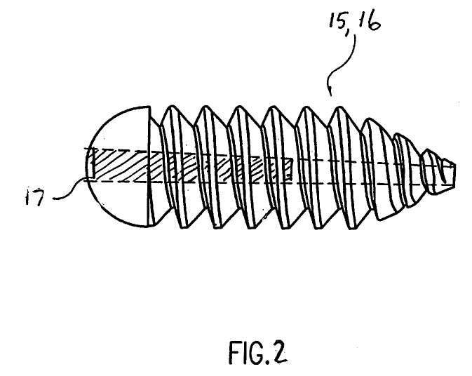

Autogenous growth factors produced from a patient's own blood, preferably contained in platelet-rich plasma (PRP) 50 obtained by a centrifugation process, are then injected into the tibial tunnel 8 and around the graft 20. Preferably, the platelet-rich plasma (PRP) 50 is injected by a surgeon during the surgery through the tibia 10 and through the cannulation of interference screw 15. Prior to the insertion of the platelet-rich plasma (PRP) 50 into the tunnel 8, interference screw 16 is plugged (with a plug 17 as shown in FIG. 2). Subsequent to the injection of the platelet-rich plasma (PRP) 50 through the portal A and into the tibial tunnel 8, cannulated interference screw 15 is also plugged with a plug 17 to prevent leaking of the platelet-rich plasma (PRP) 50 from the tunnel 8.

By providing the platelet-rich plasma (PRP) 50 within the tibial tunnel 8, healing of the operative site and bone growth are accelerated. The fixation devices (i.e., interference screws 15 and 16) prevent leakage of the the platelet-rich plasma (PRP) 50 that promotes the growth of bone and/or tissue structure and the overall healing process. The fixation devices are also simultaneously used to fix the graft in the tunnel to stabilize placement of the graft during the healing process and return to normal activity.

If the graft 20 is secured in the femur with an interference screw, instead of a transverse pin, the platelet-rich plasma (PRP) 50 containing growth factor can also be inserted into the femoral socket through the cannulation of the femoral interference screw, and the femoral screw subsequently plugged, in a similar manner to that done in the tibial side (fixation of the graft in the socket on the femoral side is performed through the tibial tunnel and precedes fixation of the graft on the tibial side).

Optionally, the platelet-rich plasma (PRP) 50 may comprise additional lubricants and/or an antiseptic chemical and/or an antibiotic. In this case, other solution excipients such as buffer salts, sugars, anti-oxidants and preservatives to maintain the bioactivity of the platelet-rich plasma (PRP) 50 and a proper pH of the plasma may be also employed. The additional lubricants and/or the antiseptic and/or the antibiotic will typically be present in the plasma in a predetermined concentration range, which will be dependent upon the particular bone site and application, as well as the specific activity of the antiseptic and/or the antibiotic.

Although the above embodiments have been described above with reference to the platelet-rich plasma (PRP) 50 provided at a particular tissue repair site, such as the tibial tunnel 8, the invention is not limited to this exemplary embodiment. Accordingly, the present invention has applicability to the injection of platelet-rich plasma and autogenous growth factors to a variety of tunnels and sockets provided within repair sites corresponding to bone, soft tissue or osteochondral tissue, among others.

The above description and drawings illustrate preferred embodiments which achieve the objects, features and advantages of the present invention. It is not intended that the present invention be limited to the illustrated embodiments. Any modification of the present invention which comes within the spirit and scope of the following claims should be considered part of the present invention.

What is claimed is:

1. A method of conducting ligament reconstruction, comprising the steps of:
providing a tunnel within a target tissue;
providing a graft within the tunnel; and
inserting growth factors into the tunnel.

2. The method of claim 1, wherein the growth factors are autogenous growth factors.

3. The method of claim 2, wherein the autogenous growth factors are contained in platelet-rich plasma.

4. The method of claim 3, wherein the platelet-rich plasma is obtained by centrifugation of blood.

5. The method of claim 2, wherein the autogenous growth factors inserted into the tunnel are supplemented with a component selected from the group consisting of lubricants, antiseptics and antibiotics.

6. The method of claim 2, further comprising the step of containing the autogenous growth factors within the tunnel.

7. The method of claim 6, further comprising the steps of:
securing a fixation device at each end of the tunnel; and
injecting the autogenous growth factors within the tunnel and through at least one end of the tunnel.

8. The method of claim 7, wherein the tunnel is a bone tunnel and the fixation device secured at each end of the tunnel is an interference screw.

9. The method of claim 8, wherein the interference screw at each end of the tunnel is cannulated, and further comprising the step of providing a plug in the interference screw at each end of the tunnel to contain the autogenous growth factors within the tunnel.

10. The method of claim 1, wherein the target tissue is bone.

11. The method of claim 1, wherein the target tissue is soft tissue.

12. The method of claim 1, wherein the target tissue is osteochondral tissue.

13. The method of claim 1, wherein the tunnel is a tibial tunnel.

14. A method of promoting bone growth, comprising the steps of:
forming a bone tunnel within a bone of a patient;
inserting a graft or ligament within the bone tunnel;
securing both ends of the graft or ligament to the bone tunnel with fixation devices; inserting autogenous growth factors into the bone tunnel; and
containing the autogenous growth factors within the bone tunnel.

15. The method of claim 14, wherein the fixation devices are cannulated and the step of containing the autogenous growth factors within the bone tunnel comprises plugging the fixation devices to prevent leaking of the autogenous growth factors from the bone tunnel.

16. The method of claim 14, wherein the autogenous growth factors are contained in platelet-rich plasma.

17. The method of claim 16, wherein the platelet-rich plasma is obtained by centrifugation of blood from the patient.

18. The method of claim 16, wherein the autogenous growth factor contained in the platelet-rich plasma is supplemented with a component selected from the group consisting of lubricants, antiseptics and antibiotics.

19. The method of claim 14, wherein at least one of the plurality of fixation devices is a cannulated interference screw.

20. The method of claim 14, wherein at least one of the fixation devices is a cannulated bioabsorbable interference screw.
